(19) United States
(12) Patent Application Publication
(10) Pub. No.: US 2022/0249061 A1
(43) Pub. Date: Aug. 11, 2022

(54) SYSTEM AND METHOD FOR ASSISTED ULTRASOUND SHEAR WAVE LASTOGRAPHY

(71) Applicant: KONINKLIJKE PHILIPS N.V., EINDHOVEN (NL)

(72) Inventors: Carolina Amador Carrascal, Everett, MA (US); Claudia Errico, Cambridge, MA (US); Grzegorz Toporek, Cambridge, MA (US); Shyam Bharat, Arlington, MA (US); Raghavendra Srinivasa Naidu, Auburndale, MA (US); Hua Xie, Cambridge, MA (US); Faik Can Meral, Mansfield, MA (US); Haibo Wang, Melrose, MA (US)

(21) Appl. No.: 17/617,338

(22) PCT Filed: Jun. 5, 2020
(86) PCT No.: PCT/EP2020/065618

§ 371 Date: Dec. 8, 2021

Related U.S. Application Data
(60) Provisional application No. 62/860,007, filed on Jun. 11, 2019.

(51) Int. Cl. A61B 8/08; A61B 8/00; G06V 10/40; G06V 10/25; G06T 7/00; G06T 11/00; G06V 10/764; G06T 7/10

(52) U.S. Cl. CPC A61B 8/485; G06V 2201/03; A61B 8/463; A61B 8/469; A61B 8/4254; G06V 10/40; G06V 10/25; G06T 7/0012; G06T 11/00; G06V 10/764; G06T 7/10; G06T 2207/10132; G06T 2207/20092; G06T 2200/24; G06T 2207/30004; A61B 8/4488

(57) ABSTRACT

A system (100) and method (2000): specify image features (1342/1344/1346) which are to be avoided in selecting a region of interest (10) in tissue of a body for making a shear wave elasticity measurement of the tissue; receive (2020) one or more image signals from an acoustic probe (120) produced from acoustic echoes from an area of the tissue; process (2030) acoustic images (504/506/508) in real-time to identify the image features which are to be avoided in selecting the region of interest; provide (2040) visual feedback to a user to choose a location for the region of interest based on the identified image features; select (2050) the region of interest in response to the visual feedback; and make (2060) the shear wave elasticity measurement of the tissue within the selected region of interest using one or more acoustic radiation force pulses.

### TECHNICAL FIELD

This invention pertains to acoustic (e.g., ultrasound) shear wave electrography, and in particular a system, device and method for assisting a user in performing ultrasound shear wave electrography.

### BACKGROUND AND SUMMARY

Acoustic (e.g., ultrasound) imaging systems are increasingly being employed in a variety of applications and contexts.

For example, shear wave elastography imaging (SWEI) has been employed to quantify tissue elasticity or stiffness that is shown to correlate with tissue pathological state. In SWEI, an ultrasonic beam applies force remotely to a region of tissue within the body of the patient (acoustic radiation force; also referred to as “push” pulse(s)). The acoustic radiation force or push pulses can be applied in such a way that elastic properties of the tissue may be measured. For example, the deformation caused by the acoustic radiation force or push pulses can be used as a source of shear waves propagating laterally away from the deformed region, which may then be imaged to interrogate adjacent regions for their material properties through time-domain shear wave velocity imaging.

In ultrasound SWEI, the pulse sequence which is employed for stiffness image generation and reconstruction is quite different from the pulse sequence for conventional brightness mode (B-mode) imaging. A typical SWEI pulse sequence consists of a long acoustic radiation force pulse (push pulse or push beam) for shear wave generation in the tissue whose elasticity is being measured, followed by conventional pulse-echoes for shear wave tracking and subsequent stiffness reconstruction in a region of interest (ROI) of the tissue.

FIG. 1 illustrates examples of the acoustic radiation force pulse and shear wave propagation in a homogeneous material. FIG. 1 shows an image 2 of an acoustic radiation force pulse employed to generate shear waves inside tissue of interest. The resulting shear wave propagation at times 2 ms and 4 ms is tracked with conventional echo images 4 and 6, respectively.

The main clinical application of SWEI has been, and still is, liver fibrosis staging. Although various commercial elastography features have been developed by multiple ultrasound vendors, each feature has its own measurement principle, reconstruction method, and outcome, creating vendor-dependent measurement in clinical settings. The main challenge of SWEI is to find corresponding thresholds to stage liver fibrosis, with large variability in measurements from one exam to anther and across vendor platforms. A general observation is that SWEI scanning experience is needed to obtain reproducible elasticity readings.

When acoustic image features resulting from acoustic reverberation (e.g. at a liver capsule boundary), acoustic shadowing (e.g. rib shadowing in liver imaging), and/or large blood vessels are in the path of the push beam, as well as in the shear wave imaging ROI, both shear wave generation and stiffness reconstruction may be highly compromised, resulting in poor measurement repeatability. In addition, robust and reproducible stiffness measurement are challenging in the presence of external motion such as a user's hand motion, a subject's bulk body motion, and physiological motion (e.g., breathing).

Due to these factors, localization of the stiffness image area or ROI is crucial for robust and reproducible stiffness measurements. Although mitigation strategies exist, such as quality control via confidence maps and constant training of users on guidelines, proper localization of the ROI would greatly improve the outcome of SWEI. Because it is difficult to find a “ground truth” for stiffness quantification, identifying the optimal place to localize the ROI based on a stiffness outcome is a challenge. However, optimal choice of the ROI is important because it has a direct impact on the clinical utility of the measured stiffness value.

Accordingly, it would be desirable to provide a system and a method which can address these challenges in SWEI. In particular, it would be desirable to provide a system and method which can assist clinicians to select a location for a region of interest within the tissue for making the shear wave elasticity measurement of the tissue, based on excluding image features—such as blood vessels, a liver capsule boundary, and ribs in the case of liver tissue, and image effects caused by significant external motion—which should be avoided in selecting the region of interest.

In one aspect of the invention, a system comprises: an acoustic probe having an array of acoustic transducer elements; and an acoustic imaging instrument connected to the acoustic probe. The acoustic imaging instrument is configured to provide transmit signals to least some of the acoustic transducer elements to cause the array of acoustic transducer elements to transmit one or more acoustic radiation force pulses to a region of interest within tissue of a body, the one or more acoustic radiation force pulses having sufficient energy to generate shear waves in the tissue. The acoustic imaging instrument is further configured to produce acoustic images of the region of interest in response to acoustic echoes received by the acoustic probe from the region of interest. The acoustic imaging instrument includes: a user interface including at least a display device; a communication interface configured to receive one or more image signals from the acoustic probe produced from the acoustic echoes from the region of interest; and a processor, and associated memory. The processor and associated memory are configured to: process the acoustic images in real-time to identify image features which are specified by the system to be avoided in selecting the region of interest for making a shear wave elasticity measurement of the tissue; provide visual feedback via the user interface to a user to choose a location for the region of interest based on the identified image features which are specified by the system to be avoided in selecting the region of interest for making the shear wave elasticity measurement of the tissue; in response to the visual feedback, select the region of interest for making the shear wave elasticity measurement of the tissue; and make the shear wave elasticity measurement of the tissue within the selected region of interest, using the one or more acoustic radiation force pulses.

In some embodiments, the acoustic images which are processed to identify the image features which are specified by the system to be avoided in selecting the region of interest for making the shear wave elasticity measurement of the tissue are shear wave elasticity images produced in response to the one or more acoustic radiation force pulses.

In some embodiments, the processor is configured to employ a neural network algorithm to process the acoustic images in real-time to identify the image features which are specified by the system to be avoided in selecting the region of interest for making the shear wave elasticity measurement of the tissue.

In some embodiments, providing visual feedback via the user interface to a user to choose the location for the region of interest includes: overlaying the acoustic images with graphical objects to identify a candidate region of interest and to show bounding boxes surrounding the image features which are specified by the system to be avoided in selecting the region of interest for making the shear wave elasticity measurement of the tissue; and providing the visual feedback via the user interface to the user to adjust a location of the candidate region of interest to avoid including the identified image features within the candidate region of interest.

In some embodiments, the processor is configured to cause the display device to display in real time a graphical object indicating a suggested adjustment for the candidate region of interest to better avoid the image features which are specified by the system to be avoided in selecting the region of interest for making the shear wave elasticity measurement of the tissue.

In some embodiments, the processor is further configured to classify a relationship between the candidate region of interest and the bounding boxes into a classified category among a plurality of predefined categories.

In some embodiments, the processor is further configured to provide a visual alert to the user via the user interface to suggest a change to at least one of: a position of the acoustic probe, a movement of the acoustic probe, the location of the candidate region of interest, wherein the processor is configured to select the alert based on the classified category.

In some embodiments, the processor is further configured to choose the location for the selected region of interest.

In some embodiments, the processor is further configured to: store the acoustic images in memory; generate from the stored acoustic images a shear wave elastography cineloop comprising a plurality of SWEI frames; and select an SWEI frame among the plurality of SWEI frames for making the shear wave elasticity measurement of the tissue.

In some embodiments, the processor is configured to identify in at least one SWEI frame of the shear wave elastography cineloop a plurality of stiffness quantification boxes within the selected region of interest for making the shear wave elasticity measurement of the tissue, and the display device is configured to overlay the stiffness quantification boxes on a displayed stiffness image of the SWEI frame.

In another aspect of the invention, a method comprises: specifying image features which are to be avoided in selecting a region of interest in tissue of a body for making a shear wave elasticity measurement of the tissue; receiving one or more image signals from an acoustic probe produced from acoustic echoes from an area of the tissue and generating acoustic images in response thereto; processing the acoustic images in real-time to identify the image features which are to be avoided in selecting the region of interest for making the shear wave elasticity measurement of the tissue; providing visual feedback to a user to choose a location for the region of interest based on the identified image features which are to be avoided in selecting the region of interest for making the shear wave elasticity measurement of the tissue; in response to the visual feedback, selecting the region of interest for making the shear wave elasticity measurement of the tissue; and making the shear wave elasticity measurement of the tissue within the selected region of interest using one or more acoustic radiation force pulses.

In some embodiments, the acoustic images are shear wave elasticity images in response to the one or more acoustic radiation force pulses.

In some embodiments, providing visual feedback to the user to choose the location for the region of interest includes: overlaying the acoustic images with graphical objects to identify a candidate region of interest and to show bounding boxes surrounding the image features which are to be avoided in selecting the region of interest for making the shear wave elasticity measurement of the tissue; and providing the visual feedback to the user to adjust a location of the candidate region of interest to avoid including the identified image features within the candidate region of interest.

In some embodiments, the method further comprises displaying in real time a graphical object indicating a suggested adjustment for the candidate region of interest to better avoid the image features which are to be avoided in selecting the region of interest for making the shear wave elasticity measurement of the tissue.

In some embodiments, the method further comprises classifying a relationship between the candidate region of interest and the bounding boxes into a classified category among a plurality of predefined categories.

In some embodiments, the method further comprises providing an alert to the user to suggest a change to at least one of: a position of the acoustic probe, a movement of the acoustic probe, the location of the candidate region of interest, wherein the alert is selected based on the classified category.

In some embodiments, a processor chooses the location for the selected region of interest.

In some embodiments, the method further comprises: storing the acoustic images in memory; generating from the stored acoustic images a shear wave elastography cineloop comprising a plurality of SWEI frames; and selecting a SWEI frame among the plurality of SWEI frames for making the shear wave elasticity measurement of the tissue.

In some embodiments, the method further comprises: identifying in at least one SWEI frame of the shear wave elastography cineloop a plurality of stiffness quantification boxes within the selected region of interest for making the shear wave elasticity measurement of the tissue; and overlaying the stiffness quantification boxes on a displayed image of the SWEI frame.

In some embodiments, the method further comprises: segmenting at least one of the identified image features within the acoustic images; defining a bounding box which encompasses the at least one identified image feature; and overlaying the bounding box on a display of the acoustic images.

### BRIEF DESCRIPTION OF THE DRAWINGS

FIG. 1 illustrates examples of the acoustic radiation force pulse and shear wave propagation in a homogeneous material.

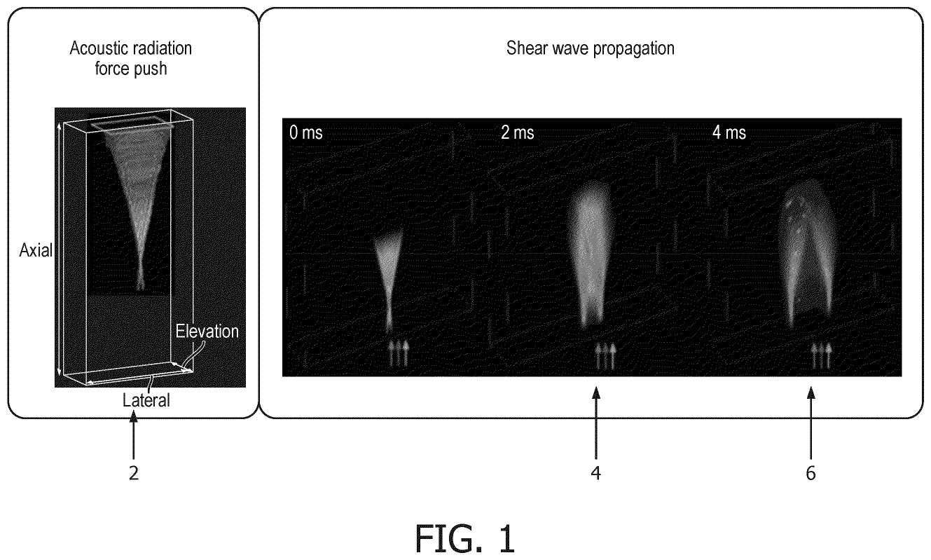

FIG. 2 illustrates an example embodiment of an acoustic imaging system which may be employed for shear wave elasticity imaging (SWEI).

FIG. 3 illustrates an example embodiment of a processing unit which may be included in an acoustic imaging apparatus.

FIG. 4 illustrates an example embodiment of an acoustic probe.

FIG. 5 illustrates examples of shear wave elasticity images which have regions of interests which are located where they include image features which should be avoided for making a shear wave elasticity measurement of the tissue.

FIG. 6 illustrates examples of images which show a region of interest (ROI) which is affected negatively by heavy respiratory motion.

FIG. 7 illustrates three different examples of shear wave elasticity images of right liver lobes accessed intercostally.

FIG. 8 illustrates examples of text alerts and corresponding actions of a user in response to the alerts.

FIG. 9 illustrates a B-image of a tissue prior to activation of elastography mode, and a shear wave elasticity image showing a candidate ROI and a proposed ROI suggested by a system and method according to some embodiments.

FIG. 10A, FIG. 10B and FIG. 10C illustrate three SWEI frames of a shear wave elastography cineloop generated from stored shear wave elasticity images of tissue.

FIG. 11 illustrates stiffness quantification boxes within an ROI in a selected SWEI frame of a shear wave elastography cineloop generated from stored shear wave elasticity images of tissue.

FIG. 12 illustrates an example architecture of a You Only Look Once (YOLO) network.

FIG. 13 illustrates a B-mode image on which feature detection has been performed and bounding boxes defined for encompassing detected features.

FIG. 14A, FIG. 14B, FIG. 14C, FIG. 14D, FIG. 14E, FIG. 14F and FIG. 14G illustrate various examples of different categories of relationships between a candidate region of interest and bounding boxes for image features which should be avoided for making a shear wave elasticity measurement of the tissue.

FIG. 15 illustrates an example embodiment of classification by a conventional neural network architecture which defines N classes based on the level of overlap between a bounding box and an ROI.

FIG. 16 illustrates an example embodiment of an algorithm with real-time visual feedback for ROI placement based on deep learning feature detection connected to a category classification network and in parallel with motion assessment.

FIG. 17 illustrates another example embodiment of an algorithm with real-time visual feedback for ROI placement based on deep learning feature detection connected to a category classification network and in parallel with motion assessment.

FIG. 18A, FIG. 18B and FIG. 18C illustrate three SWEI frames of a shear wave elastography cineloop generated from stored shear wave elasticity images of tissue, overlaid with bounding boxes for each SWEI frame.

FIG. 19 illustrates stiffness quantification box (Q-box) localization within an ROI in tissue.

FIG. 20 illustrates a flowchart of an example embodiment of a method of making a shear wave elasticity measurement of tissue.

### DETAILED DESCRIPTION

As discussed above, shear wave elastography imaging (SWEI) has been employed to quantify tissue elasticity that is shown to correlate with tissue pathological state. However, it would be desirable to provide a system and method which can assist clinicians to select a location for a region of interest within the tissue for making a shear wave elasticity measurement of the tissue, based on excluding image features—such as blood vessels, a liver capsule boundary, lesions, and ribs in the case of liver tissue—which should be avoided in selecting the region of interest.

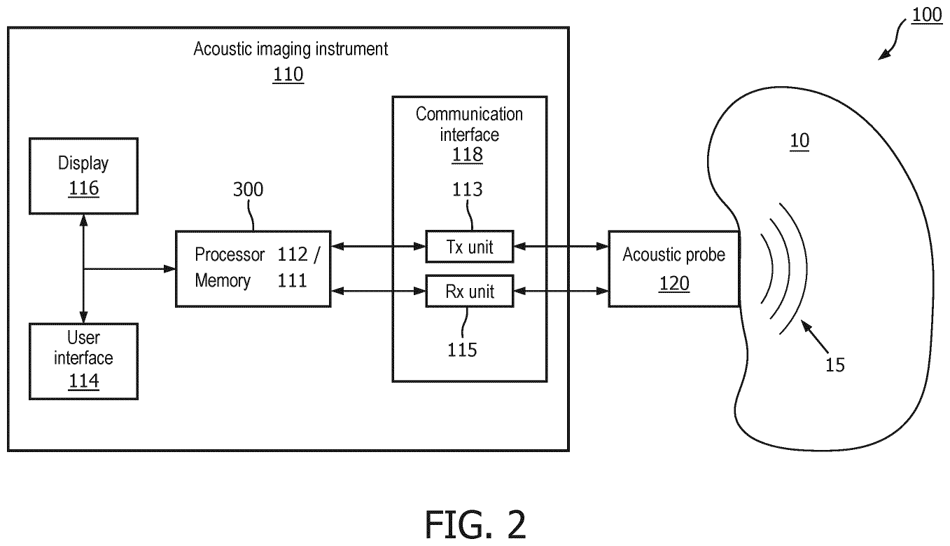

FIG. 2 illustrates an example embodiment of an acoustic imaging system 100 which may be employed for SWEI. Acoustic imaging system 100 includes an acoustic imaging instrument 110 and an acoustic probe 120. Acoustic imaging instrument 110 includes a processing unit 300, a user interface 114, a display device 116 and a communication interface 118. Processing unit 300 may include a processor 112 and a memory 111.

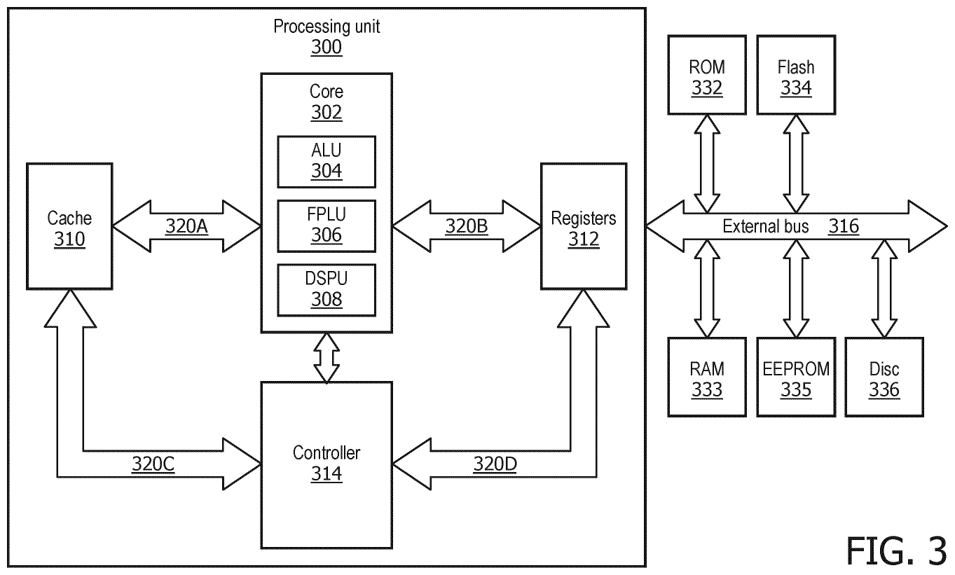

FIG. 3 is a block diagram illustrating an example processing unit 300 according to embodiments of the disclosure. Processing unit 300 may be used to implement one or more processors described herein, for example, processor 112 shown in FIG. 2. Processing unit 300 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

Processing unit 300 may include one or more cores 302. Core 302 may include one or more arithmetic logic units (ALU) 304. In some embodiments, core 302 may include a floating point logic unit (FPLU) 306 and/or a digital signal processing unit (DSPU) 308 in addition to or instead of the ALU 304.

Processing unit 300 may include one or more registers 312 communicatively coupled to core 302. Registers 312 may be implemented using dedicated logic gate circuits (e.g., flip-flops) and/or any memory technology. In some embodiments the registers 312 may be implemented using static memory. The register may provide data, instructions and addresses to core 302.

In some embodiments, processing unit 300 may include one or more levels of cache memory 310 communicatively coupled to core 302. Cache memory 310 may provide computer-readable instructions to core 302 for execution. Cache memory 310 may provide data for processing by core 302. In some embodiments, the computer-readable instructions may have been provided to cache memory 310 by a local memory, for example, local memory attached to external bus 316. Cache memory 310 may be implemented with any suitable cache memory type, for example, metal-oxide semiconductor (MOS) memory such as static random access memory (SRAM), dynamic random access memory (DRAM), and/or any other suitable memory technology.

Processing unit 300 may include a controller 314, which may control input to the processor 300 from other processors and/or components included in a system (e.g., acoustic imaging system 100 in FIG. 2) and/or outputs from processing unit 300 to other processors and/or components included in the system (e.g., communication interface 118 shown in FIG. 2). Controller 314 may control the data paths in the ALU 304, FPLU 306 and/or DSPU 308. Controller 314 may be implemented as one or more state machines, data paths and/or dedicated control logic. The gates of controller 314 may be implemented as standalone gates, FPGA, ASIC or any other suitable technology.

Registers 312 and the cache 310 may communicate with controller 314 and core 302 via internal connections 320A, 320B, 320C and 320D. Internal connections may be implemented as a bus, multiplexor, crossbar switch, and/or any other suitable connection technology.

Inputs and outputs for processing unit 300 may be provided via a bus 316, which may include one or more conductive lines. The bus 316 may be communicatively coupled to one or more components of processing unit 300, for example the controller 314, cache 310, and/or register 312. The bus 316 may be coupled to one or more components of the system, such as user interface 114 and communication interface 118 mentioned previously.

Bus 316 may be coupled to one or more external memories. The external memories may include Read Only Memory (ROM) 332. ROM 332 may be a masked ROM, Electronically Programmable Read Only Memory (EPROM) or any other suitable technology. The external memory may include Random Access Memory (RAM) 333. RAM 333 may be a static RAM, battery backed up static RAM, Dynamic RAM (DRAM) or any other suitable technology. The external memory may include Electrically Erasable Programmable Read Only Memory (EEPROM) 335. The external memory may include Flash memory 334. The external memory may include a magnetic storage device such as disc 336. In some embodiments, the external memories may be included in a system, such as ultrasound imaging system 100 shown in FIG. 2.

It should be understood that in various embodiments, acoustic imaging system 100 may be configured differently than described with respect to FIG. 2. In particular, in different embodiments, one or more functions described as being performed by elements of acoustic imaging instrument 110 may instead be performed in acoustic probe 120 depending, for example, on the level of signal processing capabilities which might be present in acoustic probe 120.

In various embodiments, processor 112 may include various combinations of a microprocessor (and associated memory), a digital signal processor, an application specific integrated circuit (ASIC), a field programmable gate array (FPGA), digital circuits and/or analog circuits. Memory (e.g., nonvolatile memory) 111, associated with processor 112, may store therein computer-readable instructions which cause a microprocessor of processor 112 to execute an algorithm to control acoustic imaging system 100 to perform one or more operations or methods which are described in greater detail below. In some embodiments, a microprocessor may execute an operating system. In some embodiments, a microprocessor may execute instructions which present a user of acoustic imaging system 100 with a graphical user interface (GUI) via user interface 114 and display device 116.

In various embodiments, user interface 114 may include any combination of a keyboard, keypad, mouse, trackball, stylus/touch pen, joystick, microphone, speaker, touchscreen, one or more switches, one or more knobs, one or more buttons, one or more lights, etc. In some embodiments, a microprocessor of processor 112 may execute a software algorithm which provides voice recognition of a user's commands via a microphone of user interface 114.

Display device 116 may comprise a display screen of any convenient technology (e.g., liquid crystal display). In some embodiments the display screen may be a touchscreen device, also forming part of user interface 114.

Communication interface 118 includes a transmit unit 113 and a receive unit 115.

Transmit unit 113 may generate one or more electrical transmit signals under control of processing unit 300 and supply the electrical transmit signals to acoustic probe 120. Transmit unit 113 may include various circuits as are known in the art, such as a clock generator circuit, a delay circuit and a pulse generator circuit, for example. The clock generator circuit may be a circuit for generating a clock signal for setting the transmission timing and the transmission frequency of a drive signal. The delay circuit may be a circuit for setting delay times in transmission timings of drive signals for individual paths corresponding to the transducer elements of acoustic probe 120 and may delay the transmission of the drive signals for the set delay times to concentrate the acoustic beams to produce acoustic probe signal 15 having a desired profile for insonifying a desired image plane. The pulse generator circuit may be a circuit for generating a pulse signal as a drive signal in a predetermined cycle.

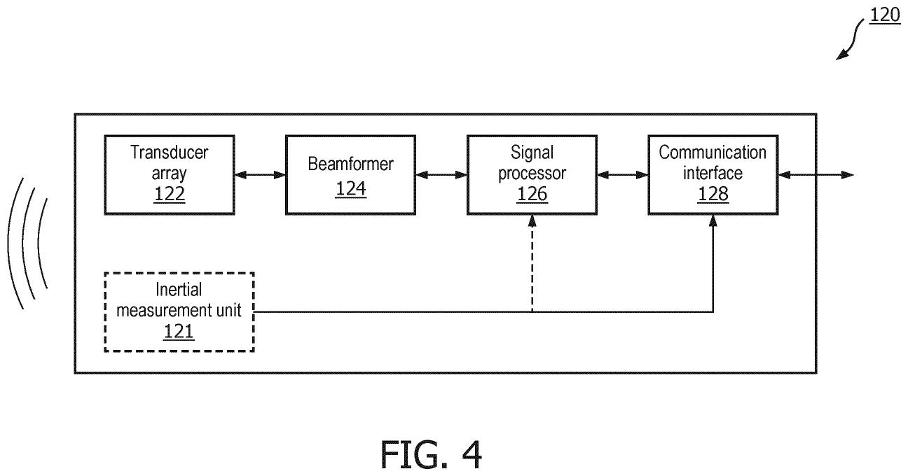

Beneficially, as described below with respect to FIG. 4, acoustic probe 120 may include an array of acoustic transducer elements 122, for example a two dimensional (2D) array or a linear or one dimensional (1D) array. For example, in some embodiments, transducer elements 122 may comprise piezoelectric elements. In operation, at least some of acoustic transducer elements 122 receive electrical transmit signals from transmit unit 113 of acoustic imaging instrument 110 and convert the electrical transmit signals to acoustic beams to cause the array of acoustic transducer elements 122 to transmit an acoustic probe signal 15 to an area of interest 10 comprising tissue, in particular human tissue, for example of a human organ such as a liver. Acoustic probe 120 may insonify an image plane in area of interest 10 and a relatively small region on either side of the image plane (i.e., it expands to a shallow field of view). In particular, acoustic probe may transmit one or more push pulses to tissue in an area of interest 10 to generate a shear wave in the tissue. Each push pulse may optionally be followed by one or more tracking pulses to interrogate the tissue of interest for imaging.

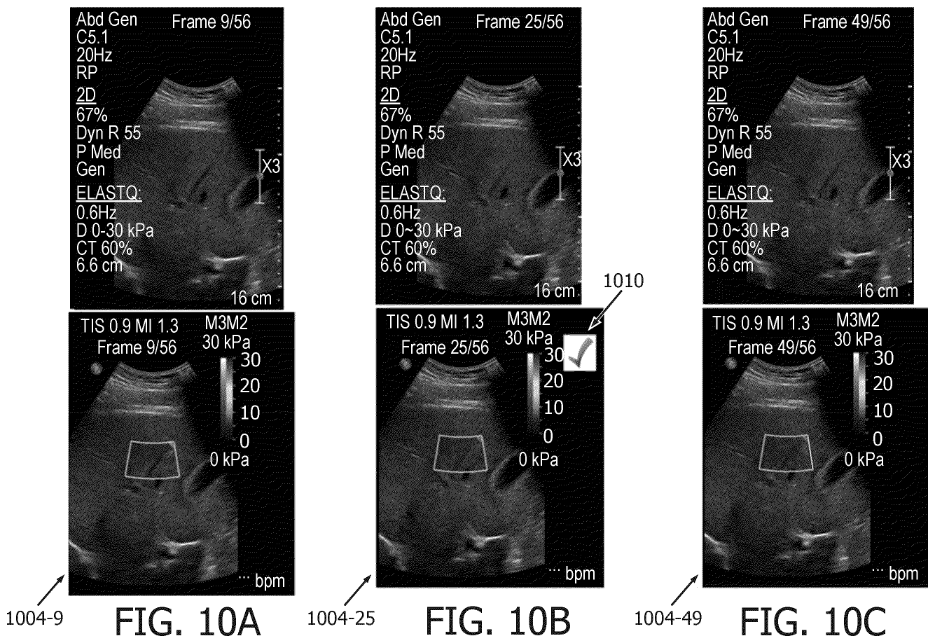

Also, at least some of acoustic transducer elements 122 of acoustic probe 120 receive acoustic echoes from area of interest 10 in response to acoustic probe signal 15 and convert the received acoustic echoes to one or more electrical signals representing an image of area of interest 10. These electrical signals may be processed further by acoustic probe 120 and communicated by a communication interface of acoustic probe 120 (see FIG. 10) to receive unit 115 as one or more image signals.

Receive unit 115 is configured to receive the one or more image signals from acoustic probe 120 and to process the image signal(s) to produce acoustic image data, including shear wave elasticity images. In some embodiments, receive unit 115 may include various circuits as are known in the art, such as one or more amplifiers, one or more A/D conversion circuits, and a phasing addition circuit, for example. The amplifiers may be circuits for amplifying the image signals at amplification factors for the individual paths corresponding to the transducer elements 122. The A/D conversion circuits may be circuits for performing analog/digital conversion (A/D conversion) on the amplified image signals. The phasing addition circuit is a circuit for adjusting time phases of the amplified image signals to which A/D conversion is performed by applying the delay times to the individual paths respectively corresponding to the transducer elements 122 and generating acoustic data by adding the adjusted received signals (phase addition). The acoustic data may be stored in memory 111 or another memory associated with acoustic imaging instrument 100.

Processing unit 300 may reconstruct acoustic data received from receiver unit 115 into an acoustic image corresponding to an image plane which intercepts area of interest 10, and subsequently causes display device 116 to display this image. The reconstructed image may for example be an ultrasound Brightness-mode “B-mode” image, otherwise known as a “2D mode” image, a “C-mode” image or a Doppler mode image, or indeed any ultrasound image.

In particular, processor 112 may reconstruct acoustic data received from receiver unit 115 into shear wave elasticity images which may be displayed on display device 116 and employed for making shear wave elasticity measurements of tissue, including in region of interest 10.

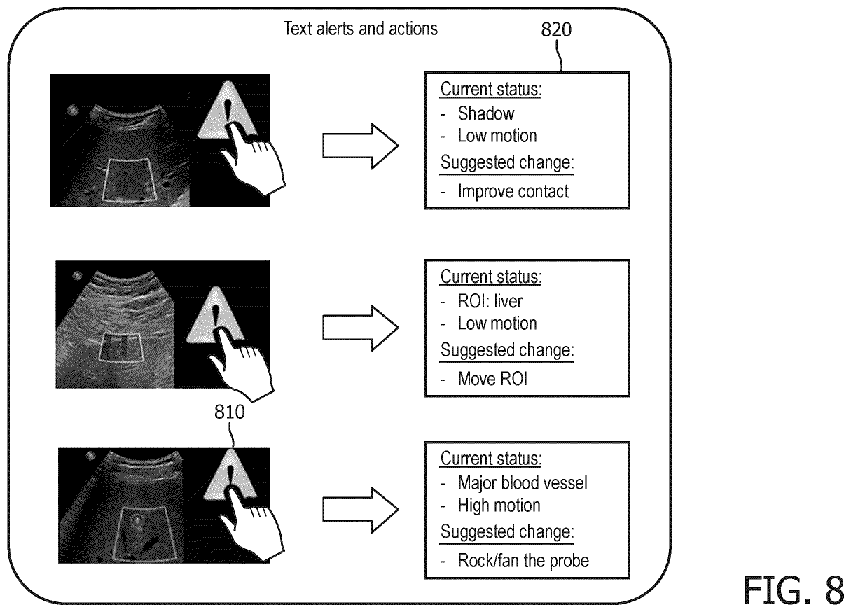

In various embodiments, processing unit 300 may execute software in one or more modules for performing one or more algorithms or methods as described below with respect to FIGS. 8-20 to assist clinicians to select a location for a region of interest within tissue for making a shear wave elasticity measurement of the tissue.

Of course it is understood that acoustic imaging instrument 110 may include a number of other elements not shown in FIG. 2, for example a power system for receiving power from AC Mains, an input/output port for communications between acoustic imaging instrument 110 and acoustic probe 120, a communication subsystem for communicating with other eternal devices and systems (e.g., via a wireless, Ethernet and/or Internet connection), etc.

In some embodiments, acoustic imaging instrument 110 also receives an inertial measurement signal from an inertial measurement unit (IMU) included in or associated with acoustic probe 120. The inertial measurement signal may indicate an orientation or pose of acoustic probe 120. The inertial measurement unit may include a hardware circuit, a hardware sensor or Microelectromechanical systems (MEMS) device. The inertial measurement circuitry may include a processor circuit running software in conjunction with a hardware sensor or MEMS device.

FIG. 4 illustrates an example embodiment of acoustic probe 120. In some embodiments, acoustic probe 120 may be configured to transmit one or more radiation force pulses, and optionally one or more tracking pulses, and receive acoustic echoes from tissue in area of interest 10.

Acoustic probe 120 includes an array of acoustic transducer elements 122, a beamformer 124, a signal processor 126, a communication interface 128, and an inertial measurement unit 121. In some embodiments, inertial measurement unit 121 may be a separate component not included within acoustic probe 120, but associated therewith, such as being affixed to or mounted on acoustic probe 120. Inertial measurement units per se are known. Inertial measurement unit 121 is configured to provide an inertial measurement signal to acoustic imaging instrument 110 which indicates a current orientation or pose of acoustic probe 120 so that a 3D volume may be constructed from a plurality of 2D images obtained with different poses of acoustic probe 120.

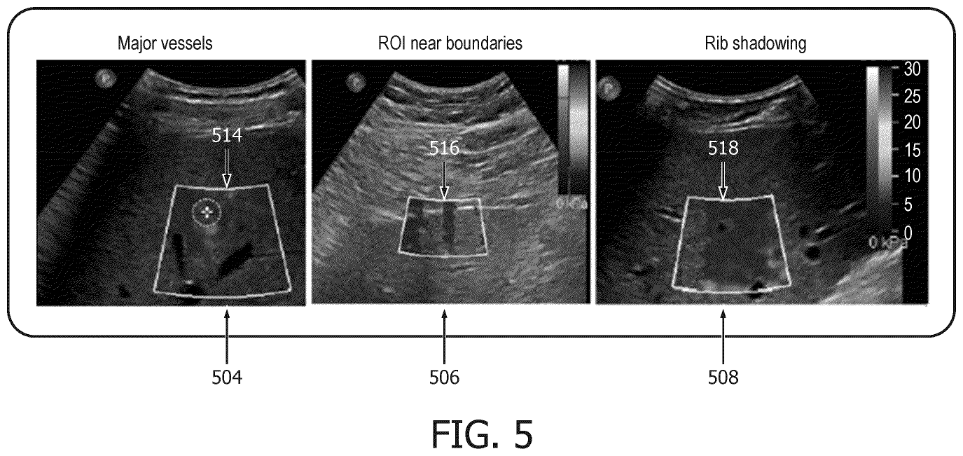

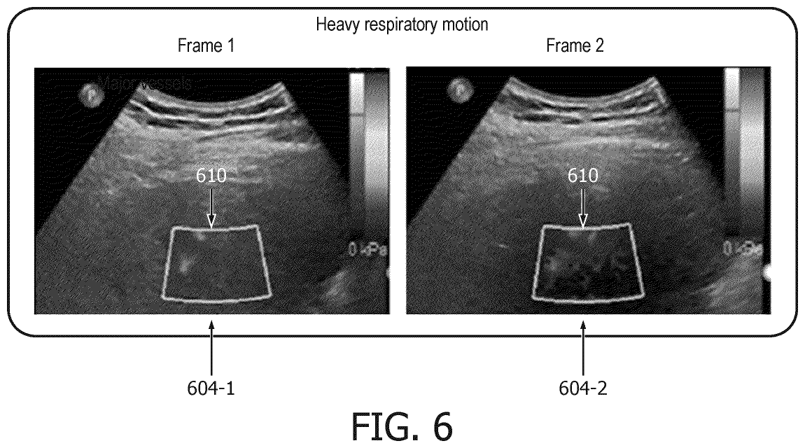

When acoustic reverberation (e.g. at liver capsule boundary), acoustic shadowing (e.g. rib shadowing in liver imaging) and large blood vessels are in the path of the push beam as well as in the shear wave imaging ROI, both shear wave generation and stiffness reconstruction are highly compromised resulting in poor measurement repeatability. In addition, robust and reproducible stiffness measurement are challenging in the presence of external motion such as user hand motion, the subject's bulk body motion, and physiological motion. FIGS. 5 and 6 illustrate examples of the aforementioned situations. Optimally, the B-mode image should be optimized for the “best acoustic window” prior to entering the elastography mode.

FIG. 5 illustrates examples of shear wave elasticity images which have regions of interests which are located where they include image features which should be avoided for making a shear wave elasticity measurement of the tissue of interest.

In particular, FIG. 5 shows on the left hand side a first shear wave elasticity image 504 where a candidate region of interest (ROI) 514, which may be selected for making a shear wave elasticity measurement of tissue of interest (e.g., liver tissue), is compromised by the inclusion of one or more long vessels. FIG. 5 also shows in the middle a second shear wave elasticity image 506 where a candidate ROI 516 is located near a liver boundary. FIG. 5 also shows on the right hand side a third shear wave elasticity image 508 where a candidate ROI 518 is compromised by rib-shadowing. In general, it would be undesirable to select any of the candidate ROIs 514, 516 or 518 as a selected ROI for making a shear wave elasticity measurement of tissue of interest (e.g., liver tissue).

FIG. 6 illustrates examples of images which show a candidate ROI which is affected negatively by heavy respiratory motion. In particular, FIG. 6 illustrates a candidate ROI 610 which changes substantially between two consecutive SWEI frames 604-1 and 604-2, evidencing that the subject is presenting respiratory motion. In general, it would be undesirable to select candidate ROI 610 as a selected ROI for making a shear wave elasticity measurement of tissue of interest (e.g., liver tissue).

Hence, localization of the stiffness image area or ROI which is selected for making a shear wave elasticity measurement of tissue of interest (e.g., liver tissue) is crucial for robust and reproducible stiffness measurements. Although mitigation strategies exist, such as quality control via confidence maps and constant training of users on guidelines, user assistance to properly localize an ROI will greatly improve the outcome of SWEI. Because it is difficult to find a ground truth for stiffness quantification, identifying the optimal place to localize the ROI based on stiffness outcome is a challenge. In addition, the end stiffness measurement may be achieved by placing a stiffness quantification Box (Q-Box) within the selected ROI that displays the majority of the uniform color. In that case, the final quantified stiffness value may be averaged over all pixels in the Q-box, and accordingly the Q-box should be in an area with minimal variance in stiffness values. This is also the reason for the importance given to optimal choice of the ROI, because it has a direct impact on the clinical utility of the measured stiffness value.

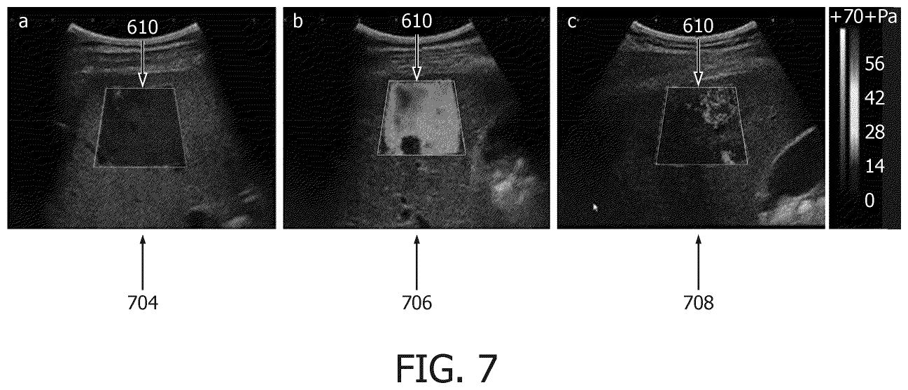

FIG. 7 illustrates three different examples of shear wave elasticity images of right liver lobes accessed intercostally. More specifically, In FIG. 7, each picture shows a background B-mode image, and inside that image a stiffness map of a candidate ROI 610 (the area inside the quasi-rectangular white box) which would normally be in color on a display screen. For each pixel inside the stiffness map, a color associated with the stiffness value at the location of the pixel is assigned. In case there is low confidence of a stiffness value at a pixel's location, then no color is assigned to that pixel and what you would see is just the background ultrasound image. “Map filling” indicates the percentage of the pixels in the stiffness map which would be colored. For example, filling a color of each pixel within the map would be 100% map filling.

FIG. 7 shows on the left hand side a first shear wave elasticity image 704 where a map of a candidate ROI 610 is relatively homogeneous, exhibiting a low spatial variability (SV) of 0.78 with high-grade elasticity map filling of 100%. FIG. 7 shows in the middle a second shear wave elasticity image 706 where the map of the candidate ROI 610 is relatively heterogeneous, exhibiting a high SV of 4.11 with a map filling of 100%. FIG. 7 shows on the right hand side a third shear wave elasticity image 708 where the map of the candidate ROI 610 exhibits a relatively heterogeneous SV of 7.88 with poor map filling of 51% (i.e., only 51% of the pixels are assigned with a stiffness value).

To address some or all of these issues, acoustic imaging system 100 may measure elasticity for tissue of interest by performing operations as described below.

Acoustic imaging system 100, for example processing unit 300, may detect main image features (e.g., blood vessels, acoustic shadowing, reverberations, lesions, tissue borders, etc.) in an acoustic image which can jeopardize shear wave imaging ROI placement and result in poor and/or irreproducible elasticity (or stiffness) measurements.

Acoustic imaging system 100, for example processing unit 300, may also provide real time text alerts to a user via user interface 114 and/or display device 116 providing visual feedback and assistance to the user during ROI placement.

FIG. 8 illustrates examples of text alerts and corresponding actions which may be performed by a user in response to these alerts.

As depicted in FIG. 8, while the user is scanning tissue of interest, an alert icon 810 ( [symbol] ) may be displayed on display device 116 if the user is not following the scanning guidelines (e.g. avoiding major vessels, avoiding rib-shadows, avoiding tissue boundaries, etc. . . . ). Once the user clicks on the alert sign via user interface 114, a text box 820 displayed on display device 116 may provide the reason for the alert, and may also propose mitigation steps (e.g. improve transducer contact, rock/fan the acoustic probe, move the candidate ROI) and provide visual feedback on bulk motion (low/mid/high motion).

Acoustic imaging system 100, for example processing unit 300, may also provide a user with a real time optimal SWEI ROI localization suggestion as the user is continuing to scan the tissue of interest. While the user is scanning tissue in B-mode using acoustic probe 120, once the user activates elastography mode via user interface 114, acoustic imaging system 100 may propose a better, or even the best/optimal, location for the shear wave imaging ROI.

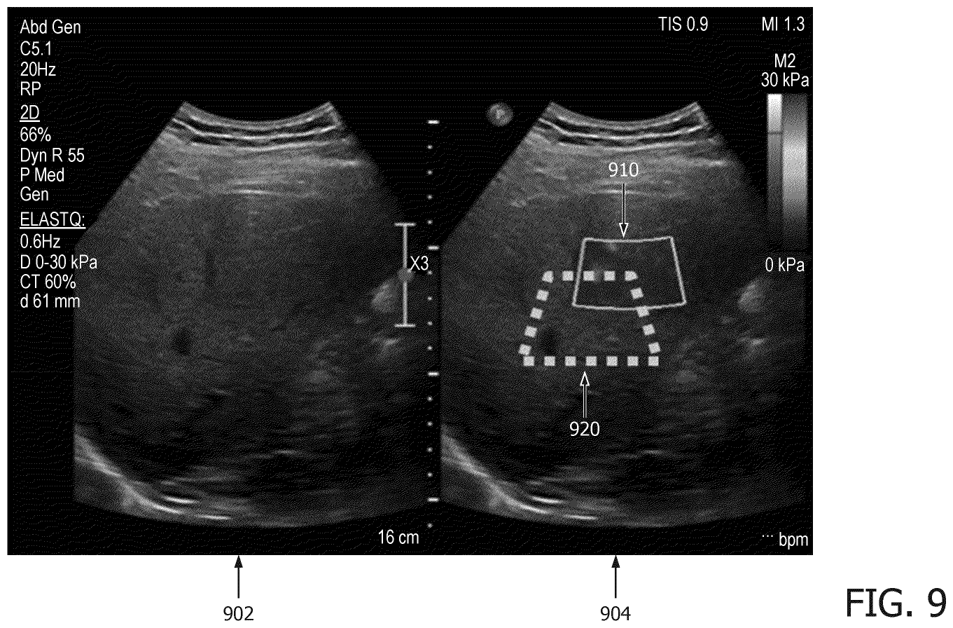

FIG. 9 illustrates on the left a B-image 902 of tissue of interest (e.g., a liver) prior to activation of elastography mode, and on the right a shear wave elasticity image 904 showing a candidate ROI 910 (which is typically a user-selected ROI), and a proposed adjusted ROI 920 which is suggested by acoustic imaging system 100 to improve or optimize elasticity measurement(s) of the tissue, for example due to the detection of blood vessels, acoustic shadowing, reverberations, lesions, tissue borders, etc. in the candidate ROI 910.

Acoustic imaging system 100, for example processing unit 300, may also generate a shear wave elastography cineloop comprised of SWEI frames produced from shear wave elasticity images obtained during a user's scan of tissue of interest with acoustic probe 120 and stored in memory. Acoustic imaging system 100 may display the shear wave elastography cineloop and/or individual SWEI frames of the shear wave elastography cineloop on display device 116, and suggest a SWEI frame with an optimal stiffness map within the shear wave elastography cineloop by highlighting the best SWEI frame.

FIG. 10A, FIG. 10B and FIG. 10C illustrate three SWEI frames of an example shear wave elastography cineloop generated from stored shear wave elasticity images of tissue. The example shear wave elastography cineloop whose SWEI frames are depicted in FIGS. 10A-10C consists of 45 SWEI frames. FIG. 10A shows a ninth SWEI frame 1004-9, FIG. 10B shows a 25^{th} SWEI frame 1004-25, and FIG. 10C shows a 49^{th} SWEI frame 1004-49. Here, SWEI frame 1004-25 is the best SWEI frame for selecting a ROI and performing an elasticity or stiffness measurement in the selected ROI, and this may be indicated to the user by annotating or overlaying the display of the SWEI frame with a check mark 1010 or other indicator, as shown in FIG. 10B.

Acoustic imaging system 100, for example processing unit 300, may also determine a good or optimal location for one or more stiffness quantification boxes (Q-boxes) within an ROI of a selected SWEI frame, and may display the recommended Q-box location(s) in the ROI on display device 116.

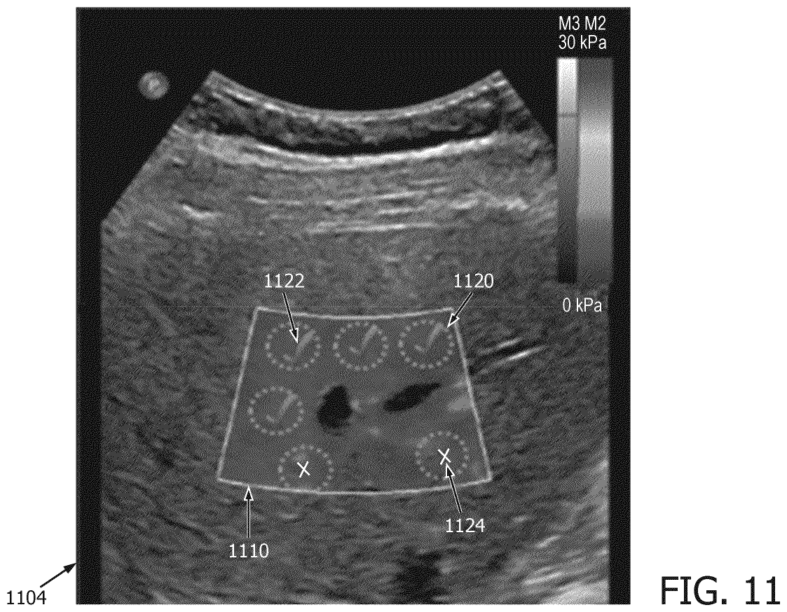

FIG. 11 illustrates Q-boxes 1120 within an ROI 1110 in a selected SWEI frame 1104 of a shear wave elastography cineloop generated from stored shear wave elasticity images of tissue. Here, the suggested or recommended optimal locations for Q-boxes 1120 are indicated by annotating or overlaying the display of SWEI frame 1104 on display device 116 with a check sign (which might be green on a color display), and the locations to avoid for Q-boxes are indicated by annotating or overlaying the display of SWEI frame 1104 on display device 116 with a cross sign (which might be red on a color display).

If/when a user indicates via user interface 114 that the indicated Q-boxes are approved, then acoustic imaging system 100 may make an elasticity measurement for the tissue using the data from inside the Q-boxes.

In accordance with the various operations described above, in various embodiments acoustic imaging system 100 may operate as follows.

Acoustic imaging system 100, and in particular processing unit 300, may perform object detection on a B-mode acoustic image. Convolutional neural networks (CNNs) can be adapted to provide real-time, per-frame object detection. A You-Only-Look-Once (YOLO) network, or other neural network, such as Regional-CNN (R-CNN), Mask-R-CNN or Faster R-CNN, developed for detecting objects, may have a very simple architecture and can be used for this purpose. Such networks may be implemented by processing unit 300. The detection network can simultaneously localize and recognize many B-mode image features such as large blood vessels, shadowing, nodules, lesions, surrounding tissue and tissue boundaries. The output of the network may be one or more of the classes of objects and corresponding probabilities and their respective locations (e.g., in the form of bounding boxes around the objects).

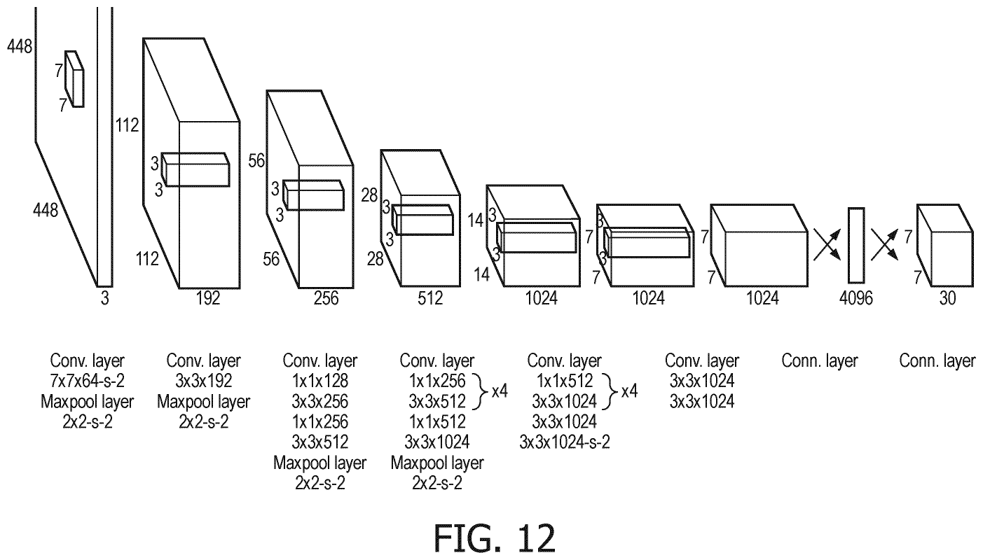

FIG. 12 illustrates an example architecture of a You Only Look Once (YOLO) network 1200. In some embodiments, YOLO network 1200 may be implemented by processing unit 300 of acoustic imaging system 100. Details of the operation of YOLO network 1200 for feature detection in images are known to those skilled in the art.

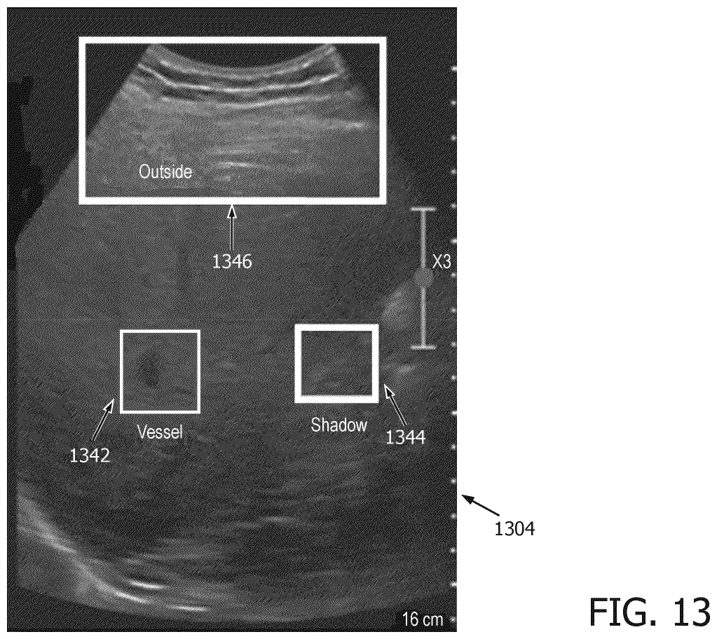

FIG. 13 illustrates a B-mode image 1304 on which image feature detection by an object detection network, such as object detection network 1200, has been performed, and bounding boxes 1342, 1344 and 1346 have been defined for encompassing detected image features to be avoided when locating an ROI for making an elasticity measurement. In particular, bounding box 1342 encompasses a detected blood vessel, bounding box 1344 encompasses an area shadowed, for example by one or more ribs, and bounding box 1346 encompasses an area of tissue outside the tissue of interest (e.g., liver tissue).

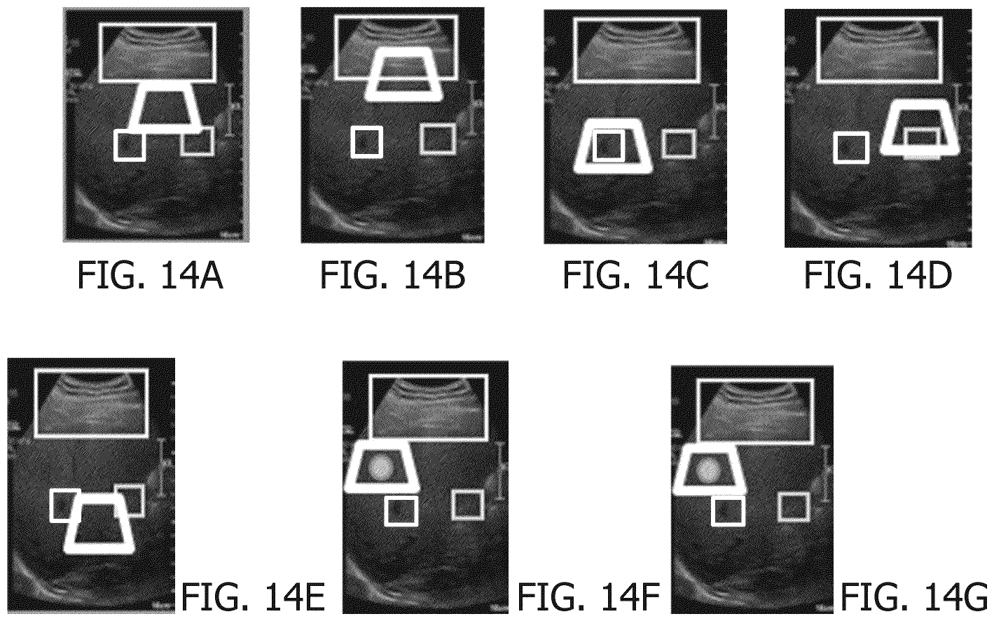

In some embodiments, acoustic imaging system 100, and in particular processing unit 300, may also employ a neural network architecture or a rule-based classifier to classify the object detection network output (e.g., bounding boxes) and the current candidate ROI (typically selected by the user). For instance, a classification system with seven different categories of relationships is illustrated in FIGS. 14A-14G.

More specifically, FIGS. 14A-14G illustrate seven examples of relationships between candidate regions of interest, and bounding boxes for features which should be avoided for making a shear wave elasticity measurement of the tissue. Each of the relationships shown in in FIGS. 14A-14G belongs to a different corresponding category among a predefined set of categories employed by acoustic imaging system 100.

Associated with each of these categories may be an alert and a recommendation or suggestion to a user, which may be presented to the user as an icon and/or text message via display device 114.

Table 1 below illustrates examples of category definitions, alerts, and recommendations which may be associated with the seven different categories illustrated by the examples of FIGS. 14A-14G.

TABLE 1

| Category | Definition | Illustration | Alert Icon and/or Text |
|---|---|---|---|
| 0 | Detected objects and user selection | FIG. 14A | No Alert |
| | of ROI) don't overlap | | |
| 1 | Candidate ROI overlaps bounding | FIG. 14B | Alert: ROI is outside tissue of |
| | box 1346 | | interest. |
| | | | Suggestions: Move the ROI |
| 2 | Candidate ROI overlaps bounding | FIG. 14C | Alert: Major vessel in ROI |
| | box 1342 | | Suggestion: Rock/fan the |
| | | | probe |
| 3 | Candidate ROI overlaps bounding | FIG. 14D | Alert: Shadowing in ROI |
| | box 1344 | | Suggestion: Improve |
| | | | transducer contact |
| 4 | Candidate ROI is near bounding | FIG. 14E | Alert: Vessel near ROI |
| | box 1346 | | Suggestion: Rock/fan the |
| | | | probe |
| 5 | Candidate ROI is overlapping a | FIG. 14F | Alert: Nodule or suspicious |
| | bounding box corresponding to a | | area in the field of view |
| | nodules or suspicious region | | Action: Show nodule |
| 6 | Nodule or suspicious region is | FIG. 14G | Alert: Nodule or suspicious |
| | detected, but candidate ROI does | | area present, but not captured |
| | not contain the nodule/suspicious | | by ROI |
| | region | | Suggestion: Move ROI to |
| | | | cover nodule, if nodule |
| | | | stiffness quantification is |
| | | | desired |

For a simple classification problem using the seven classes described in Table 1, a simple rule-based algorithm can be employed. In this case, information of the precise level or percentage of overlap between a candidate ROI (which has typically been selected by a user) and one or more bounding boxes, and the location of the overlap, is not fully employed.

However, in the case of a more detailed and complex classification, precise information on the percentage of overlap and the location may be employed. In that case, a conventional CNN can be employed by acoustic imaging system 100, where the input is a generated color map containing color-coded boxes corresponding to the candidate ROI and the bounding boxes, and the output are more specific classes. For example, Class 1 in Table 1 can be expanded to multiple subclasses where, for each subclass, bounding box 1346 and the candidate ROI overlap at different levels or percentages and/or at different locations.

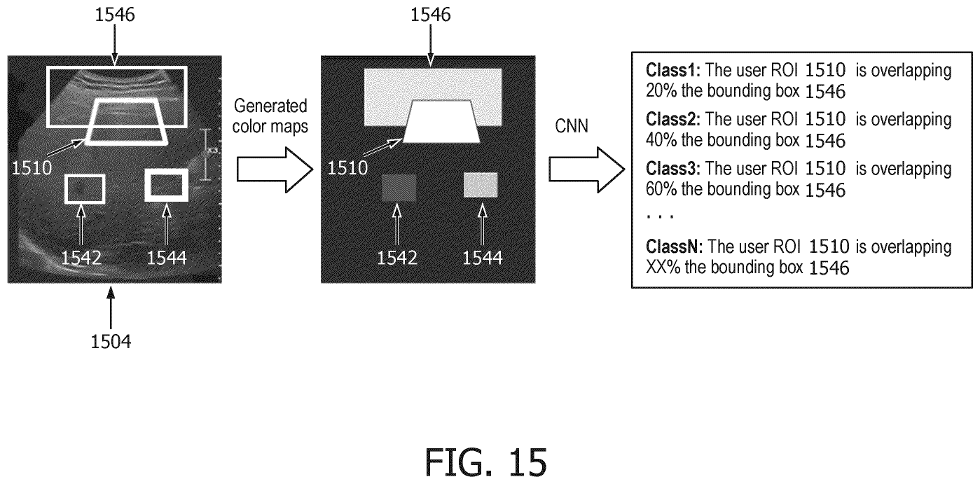

FIG. 15 illustrates an example embodiment of classification by a conventional neural network architecture which defines N classes based on the level or percentage of overlap between a bounding box and a candidate ROI. As depicted in FIG. 15, maps (which would typically be in color on a color display) are generated from a candidate ROI and bounding boxes in an acoustic image 1504. Shown in FIG. 15 is an ROI 1510, a bounding box 1542 which encompasses a detected blood vessel, a bounding box 1544 which encompasses an area shadowed, for example by one or more ribs, and a bounding box 1546 which encompasses an area of tissue outside the tissue of interest (e.g., liver tissue).

Acoustic imaging system 100, and in particular processing unit 300, also may assess movement of the subject and/or a user's hand, during acquisition of an acoustic image. In some embodiments, such motion may be characterized as high, medium, or low and the qualification of an acoustic image or SWEI imaging frame of a shear wave elastography cineloop, and a candidate ROI in that image or SWEI frame, for making a tissue elasticity measurement may depend on that characterization.

In some embodiments, motion assessment may be based on one or more of:
(1) a user's hand/probe motion assessment from external sensors such as Inertial Measurement Unit (IMU) 121, optical tracking, electromagnetic tracking, fiber optic tracking, etc.;
(2) a subject's physiological motion from image-based motion tracking in consecutive b-mode images, for example by employing fast motion tracking algorithms such as registration based (MeshMe, Demons) or speckle tracking;
(3) an electrocardiogram (ECG) signal for detection and gating of cardiac motion; and
(4) external skin markers for detection and gating of respiratory motion.

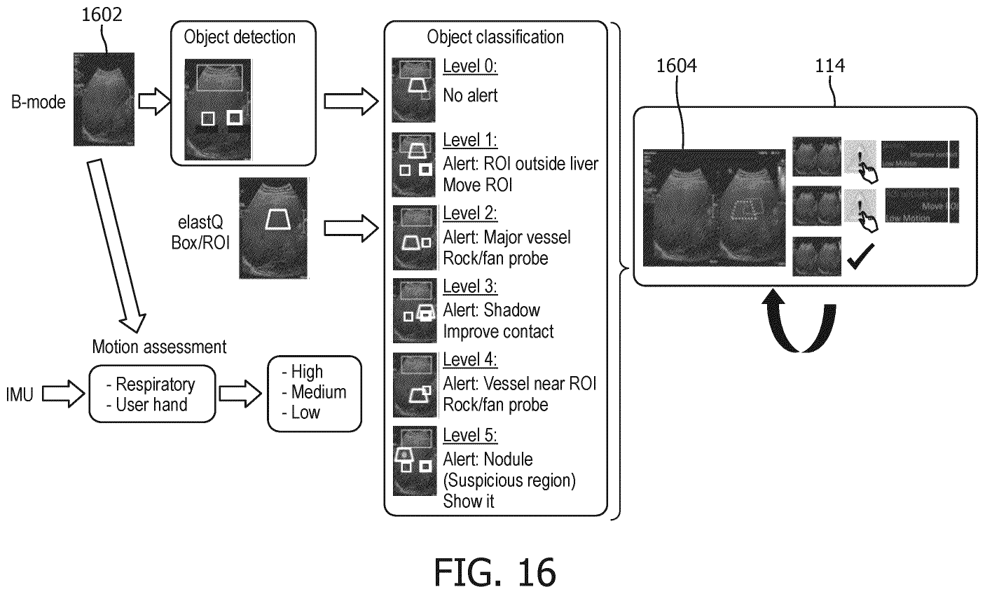

FIG. 16 illustrates an example embodiment of an algorithm with real-time visual feedback for ROI placement based on deep learning feature-detection, connected to a category classification network, and in parallel with motion assessment.

Real time b-mode images 1602 captured by acoustic imaging system 100 are input to the object detection network (e.g., implemented by processing unit 300), then the output (objects within defined bounding boxed), in combination with the user selection of a candidate ROI for elasticity (stiffness) measurements are input to a multi-category classification network that outputs, in real time, visual feedback to the user as described in Table 1 above. In parallel, a motion assessment level is also provided as visual (e.g., displayed text) feedback (high/medium/low motion) for qualifying an image or SWEI frame as appropriate for making elasticity measurements of the tissue of interest. While the user is interacting with the candidate ROI placement, if the acoustic imaging system 100 finds a level 0 classification as defined in Table 1, the acoustic imaging system 100 may indicate this ROI location to the user with dashed lines on an annotated acoustic image 1604 presented to a user via user interface 114 and displayed on display device 116, beneficially in real time as the user continues to scan the tissue of interest with acoustic probe 120.

In some embodiments, by removing user selection of a candidate ROI, and implementing an object detection network, such as object detection network 1200 described above with respect to FIGS. 12 and 13, acoustic imaging system 100 may automatically suggest a location or area (e.g., an optimal location or area) in which to locate the ROI. The ROI can also be adjustable in size and shape depending on the best ROI location.

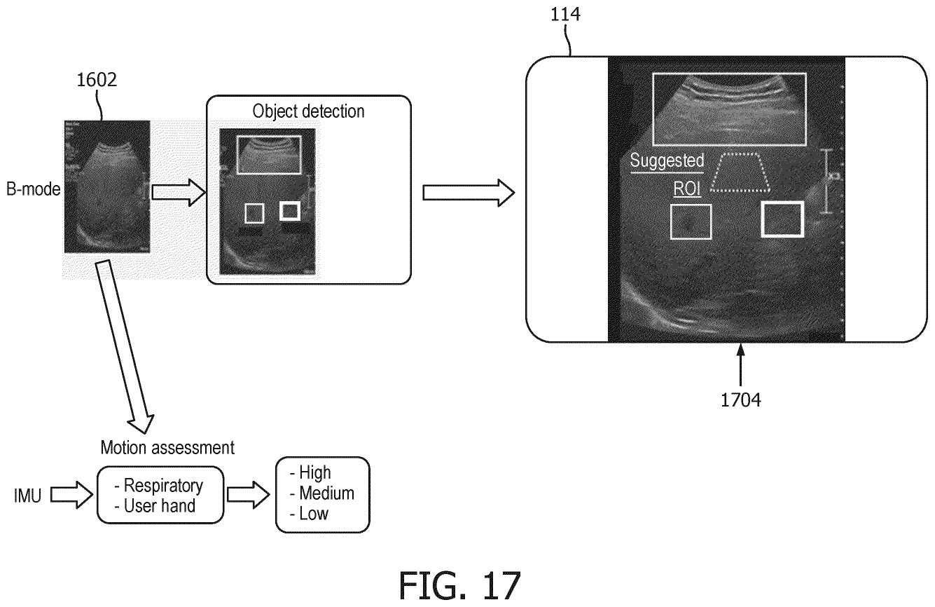

FIG. 17 illustrates an example of these embodiments which presents to the user an annotated acoustic image 1704, indicating by dashed lines a suggested location for the ROI.

In some embodiments, acoustic imaging system 100 can suggest an ROI location to a user while operating in B-mode, before the user positions an ROI in the elastography mode. If the user agrees with the system recommendation via user interface 114 (e.g., by clicking a button, touching an area of display device 116, voice command, etc.), then acoustic imaging system 100 can automatically position the ROI in that location, ready to make an elasticity measurement.

In some embodiments, during a review of the acoustic images acquired by the scan of acoustic probe 120, each SWEI frame of a shear wave elastography cineloop generated from the acquired acoustic images is applied to an object detection network, such as object detection network 1200 described above with respect to FIGS. 12 and 13. Based on the object detection network output, acoustic imaging system 100 may suggest the best SWEI frame within the shear wave elastography cineloop to perform an elasticity quantification process. The suggestion of the best SWEI frame may be based on rule-based approaches, deep learning approaches, or by simply relying on the size of the bounding boxes. For example, because shear wave elastography cineloops are videos that can be decomposed into temporal segments (single SWEI frames), acoustic imaging system 100 may additionally predict which would be the best SWEI frame to perform its elasticity measurement of the tissue of interest.

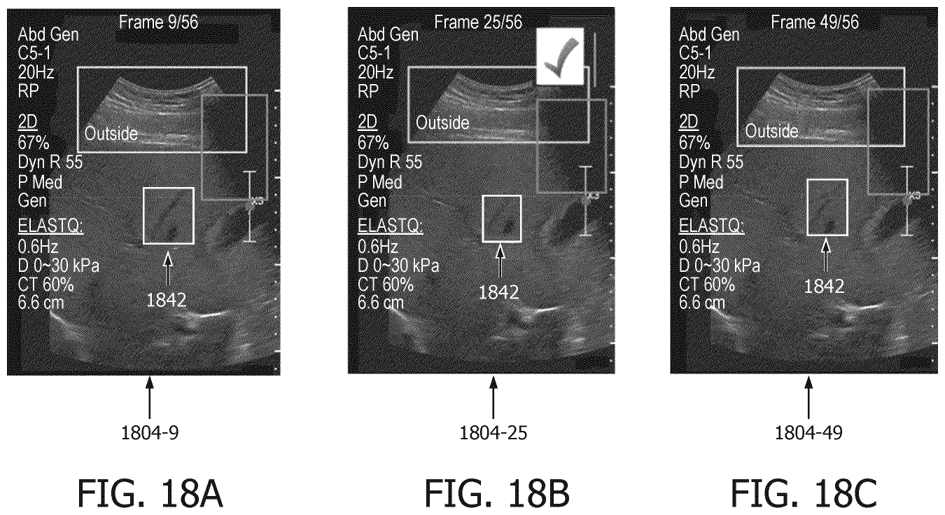

FIG. 18A, FIG. 18B and FIG. 18C illustrate three example SWEI frames of a shear wave elastography cineloop generated from stored shear wave elasticity images of tissue, overlaid with bounding boxes for each SWEI frame.

As depicted in FIGS. 18A-18C, the bounding box 1842 for the main vessel in the middle of the B-mode image gets smaller at SWEI frame 1804-25 (meaning that it is disappearing from the imaging view), while is consistently dominant and takes most of the imaging field in SWEI frames 1804-9 and 1804-49. Hence, acoustic imaging system 100 may suggest to the user that the best or optimal SWEI frame for performing the shear wave elasticity measurement would be 1804-25. The user may respond to this suggestion via user interface 114, e.g., by clicking a button, touching an area of display device 116, voice command, etc.

In some embodiments, during scan review of acquired acoustic images, an object detection network, such as object detection network 1200 described above with respect to FIGS. 12 and 13, may be applied to each acquired SWEI frame of a shear wave elastography cineloop. Based on the output of the network, acoustic imaging system 100 may suggest the best spatial areas within the ROI to perform Q-box stiffness quantification analysis, as discussed above with respect to FIG. 11.

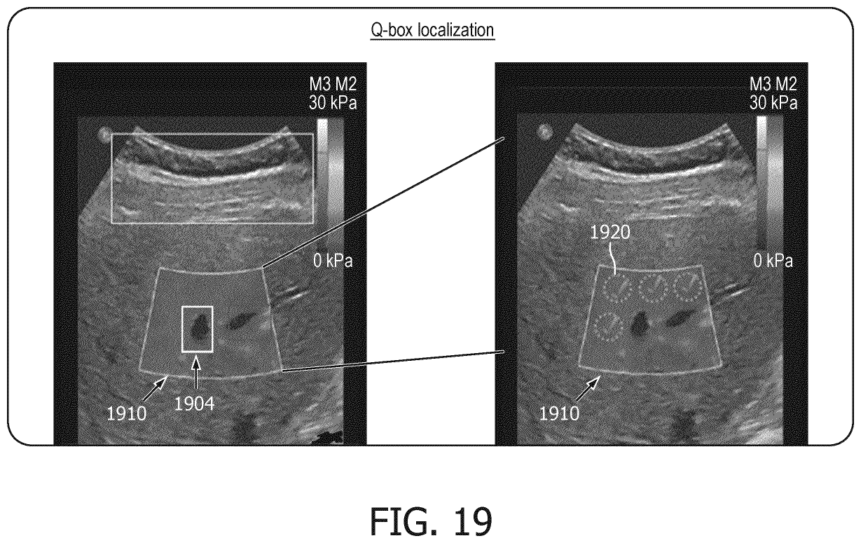

FIG. 19 illustrates an example of stiffness quantification box (Q-box) localization within an ROI in tissue. In particular, FIG. 19 illustrates an example where a bounding box 1904 for a major blood vessel is located within the selected ROI 1910. In this case, acoustic imaging system 100 suggests locating Q-boxes 1920 within ROI 1910 so that they avoid bounding box 1904.

Especially for small patients, bounding boxes for detected features to be avoided in the elasticity measurements, might cover significant part of the tissue of interest, particularly the liver parenchyma, thus leaving little space for the ROI placement.

Accordingly, in some embodiments, the detection and classification steps described above may be followed by the segmentation of the inner structures, such as vessels or shadows, so that the shape of the bounding box may be adjusted and/or the size of the bounding box reduced accordingly. Structures can be segmented using methods known in the art, such as active contours, threshold-based methods, or region growing algorithms. Alternatively, to avoid additional step segmentation and detection can be combined using deep learning type algorithm, such as Mask R-CNN that is known in the art. This network first performs semantic segmentation and embraces this binary mask with a bounding box.

As discussed above, the robustness of an SWEI imaging operation is susceptible to motion of both the probe and tissue. Although several mitigation techniques are available, such as ECG gating, external tracking devices, or IMU sensors, these techniques depend on external devices. External devices are usually bulky and not easy to integrate into existing clinical workflows.

Accordingly, in some embodiments, acoustic imaging system 100 may detect motion of the detected bounding boxes, for instance rapid changes in the size of a bounding box, and employ that detected motion to predict the motion of the tissue. As soon as the motion stabilizes, acoustic imaging system 100 may automatically position and display the ROI on the acoustic image.

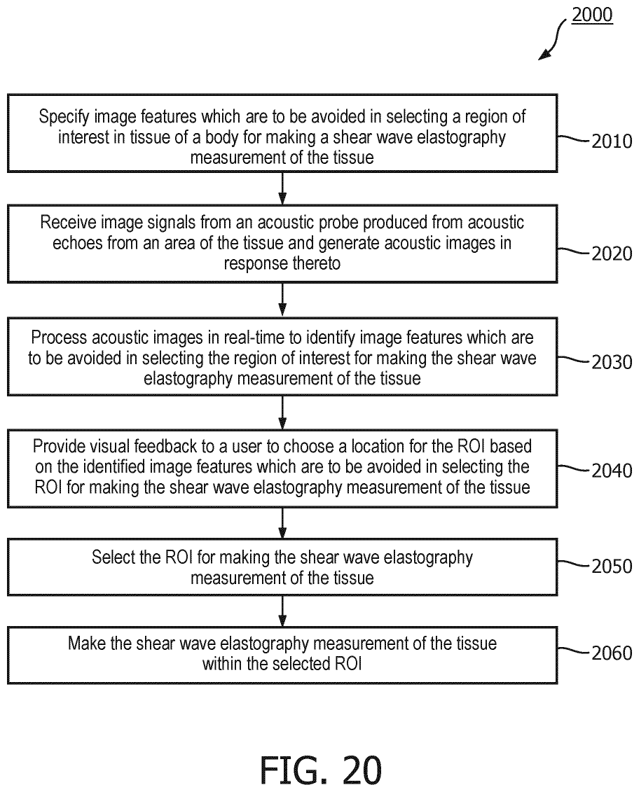

FIG. 20 illustrates a flowchart of an example embodiment of a method 2000 of making a shear wave elasticity measurement of tissue.

An operation 2010 includes specifying features which are to be avoided in selecting a region of interest in tissue of a body for making a shear wave elasticity measurement of the tissue.

An operation 2020 includes receiving one or more image signals from an acoustic probe produced from acoustic echoes from an area of the tissue and generating acoustic images in response thereto.

An operation 2030 includes processing the acoustic images in real-time to identify the features which are to be avoided in selecting the region of interest for making the shear wave elasticity measurement of the tissue.

An operation 2040 includes providing visual feedback to a user to choose a location for the region of interest based on the identified image features which are to be avoided in selecting the region of interest for making the shear wave elasticity measurement of the tissue.

An operation 2050 includes selecting the region of interest for making the shear wave elasticity measurement of the tissue, in response to the visual feedback.

An operation 2060 includes making the shear wave elasticity measurement of the tissue within the selected region of interest using one or more acoustic radiation force pulses.

It should be understood that the order of various operations in FIG. 20 may be changed or rearranged, and indeed some operations may actually be performed in parallel with one or more other operations. In that sense, FIG. 20 may be better viewed as a numbered list of operations rather than an ordered sequence.

While preferred embodiments are disclosed in detail herein, many variations are possible which remain within the concept and scope of the invention. Such variations would become clear to one of ordinary skill in the art after inspection of the specification, drawings and claims herein. The invention therefore is not to be restricted except within the scope of the appended claims.

1. A system, comprising:
an acoustic probe having an array of acoustic transducer elements; and
an acoustic imaging instrument connected to the acoustic probe and configured to provide transmit signals to least some of the acoustic transducer elements to cause the array of acoustic transducer elements to transmit one or more acoustic radiation force pulses to a region of interest within tissue of a body, the one or more acoustic radiation force pulses having sufficient energy to generate shear waves in the tissue, wherein the acoustic imaging instrument is further configured to produce acoustic images of the region of interest in response to acoustic echoes received by the acoustic probe from the region of interest, the acoustic imaging instrument including:
a user interface including at least a display device;
a communication interface configured to receive one or more image signals from the acoustic probe produced from the acoustic echoes from the region of interest; and
a processor, and associated memory, configured to:
process the acoustic images in real-time to identify image features which are specified by the system to be avoided in selecting the region of interest for making a shear wave elasticity measurement of the tissue,
provide visual feedback via the user interface to a user to choose a location for the region of interest based on the identified image features which are specified by the system to be avoided in selecting the region of interest for making the shear wave elasticity measurement of the tissue,
in response to the visual feedback, select the region of interest for making the shear wave elasticity measurement of the tissue, and
make the shear wave elasticity measurement of the tissue within the selected region of interest, using the one or more acoustic radiation force pulses.

2. The system of claim 1, wherein the acoustic images which are processed to identify the image features which are specified by the system to be avoided in selecting the region of interest for making the shear wave elasticity measurement of the tissue are shear wave elasticity images produced in response to the one or more acoustic radiation force pulses.

3. The system of claim 1, wherein the image features which are specified by the system to be avoided in selecting the region of interest for making the shear wave elasticity measurement of the tissue include at least one of tissue boundaries, vessels, shadowing, and lesions.

4. The system of claim 1, wherein the processor and associated memory are further configured to provide visual feedback via the user interface to the user to choose the location for the region of interest by:
overlaying the acoustic images with first graphical objects to identify a candidate region of interest and to show bounding boxes surrounding the image features which are specified by the system to be avoided in selecting the region of interest for making the shear wave elasticity measurement of the tissue; and
providing the visual feedback via the user interface to the user to adjust a location of the candidate region of interest to avoid including the identified image features within the candidate region of interest.

5. The system of claim 4, wherein the processor is configured to cause the display device displaying in real time a second graphical object indicating a suggested adjustment for the candidate region of interest to better avoid the image features which are specified by the system to be avoided in selecting the region of interest for making the shear wave elasticity measurement of the tissue.

6. The system of claim 4, wherein the processor is further configured to classify a relationship between the candidate region of interest and the bounding boxes into a classified category among a plurality of predefined categories.

7. The system of claim 6, wherein the processor is further configured to provide a visual alert to the user via the user interface to suggest at least one of: a change in a position of the acoustic probe, a change in a movement of the acoustic probe, a change in the location of the candidate region of interest, and reducing external motion, wherein the processor is configured to select the visual alert based on the classified category.

8. The system of claim 1, wherein the processor is further configured to choose the location for the selected region of interest.

9. The system of claim 1, wherein the processor is further configured to:
store the acoustic images in the memory;
generate from the stored acoustic images a shear wave elastography cineloop comprising a plurality of shear wave elastography imaging frames; and
select a shear wave elastography imaging frame among the plurality of shear wave elastography imaging frames for making the shear wave elasticity measurement of the tissue.

10. The system of claim 9, wherein the processor is configured to identify in the selected shear wave elastography imaging frame a plurality of stiffness quantification boxes within the selected region of interest for making the shear wave elasticity measurement of the tissue, and the display device is configured to overlay the stiffness quantification boxes on a displayed shear wave elastography image of the shear wave elastography imaging frame.

11. A method, comprising:
specifying image features which are to be avoided in selecting a region of interest in tissue of a body for making a shear wave elasticity measurement of the tissue;
receiving one or more image signals from an acoustic probe produced from acoustic echoes from an area of the tissue and generating acoustic images in response thereto;
processing the acoustic images in real-time to identify the image features which are to be avoided in selecting the region of interest for making the shear wave elasticity measurement of the tissue;
providing visual feedback to a user to choose a location for the region of interest based on the identified image features which are to be avoided in selecting the region of interest for making the shear wave elasticity measurement of the tissue;
in response to the visual feedback, selecting the region of interest for making the shear wave elasticity measurement of the tissue; and
making the shear wave elasticity measurement of the tissue within the selected region of interest using one or more acoustic radiation force pulses.

12. The method of claim 11, wherein the acoustic images are shear wave elasticity images in response to the one or more acoustic radiation force pulses.

13. The method of claim 11, wherein providing visual feedback to the user to choose the location for the region of interest includes:
overlaying the acoustic images with first graphical objects to identify a candidate region of interest and to show bounding boxes surrounding the image features which are to be avoided in selecting the region of interest for making the shear wave elasticity measurement of the tissue; and
providing the visual feedback to the user to adjust a location of the candidate region of interest to avoid including the identified image features within the candidate region of interest.

14. The method of claim 13, further comprising displaying in real time a second graphical object indicating a suggested adjustment for the candidate region of interest to better avoid the image features which are to be avoided in selecting the region of interest for making the shear wave elasticity measurement of the tissue.

15. The method of claim 13, further comprising employing an inertial measurement unit associated with the acoustic probe to detect motion of the acoustic probe while the acoustic probe receives the acoustic echoes from the tissue.

16. The method of claim 13, further comprising:
classifying a relationship between the candidate region of interest and the bounding boxes into a classified category among a plurality of predefined categories; and
providing a visual alert to the user to suggest at least one of: a change in a position of the acoustic probe, a change in a movement of the acoustic probe, a change in the location of the candidate region of interest, and reduce external motion, wherein the alert is selected based on the classified category.

17. The method of claim 11, wherein a processor chooses the location for the selected region of interest.

18. The method of claim 11, further comprising:
storing the acoustic images in memory;
generating from the stored acoustic images a shear wave elastography cineloop comprising a plurality of shear wave elastography imaging frames; and
selecting a shear wave elastography imaging frame among the plurality of shear wave elastography imaging frames for making the shear wave elasticity measurement of the tissue.

19. The method of claim 18, further comprising:
identifying in at least one shear wave elastography imaging frame of the shear wave elastography cineloop a plurality of stiffness quantification boxes within the selected region of interest for making the shear wave elasticity measurement of the tissue; and
overlaying the stiffness quantification boxes on a displayed image of the shear wave elastography imaging frame.

20. The method of claim 11, further comprising:
segmenting at least one of the identified image features within the acoustic images;
defining a bounding box which encompasses the at least one segmented identified image feature; and
overlaying the bounding box on a display of the acoustic images.
